(19) United States
(12) Patent Application Publication
(10) Pub. No.: US 2020/0395125 A1
(43) Pub. Date: Dec. 17, 2020

(54) METHOD AND APPARATUS FOR MONITORING A HUMAN OR ANIMAL SUBJECT

(71) Applicant: Oxford University Innovation Limited, Oxford (GB)

(72) Inventors: David CLIFTON, Oxford (Oxfordshire) (GB); Chris PUGH, Horton-cum-Studley (Oxfordshire) (GB); Tingting ZHU, Oxford (Oxfordshire) (GB); Glen Wright COLOPY, Cary, NC (US)

(21) Appl. No.: 16/977,491

(22) PCT Filed: Jan. 16, 2019
(86) PCT No.: PCT/GB2019/050113

§ 371 Date: Sep. 2, 2020

(30) Foreign Application Priority Data
Mar. 5, 2018 (GB) 1803506.3

(51) Int. Cl. G16H 50/20; A61B 5/0205; A61B 5/00; G16H 40/60

(52) U.S. Cl. CPC G16H 50/20; A61B 5/02055; A61B 5/7203; A61B 5/021; A61B 5/7267; G16H 40/60; A61B 5/7246

(57) ABSTRACT

Methods and apparatus for monitoring a human or animal subject are disclosed. In one arrangement, test data representing a time-series of physiological measurements performed on a subject in a measurement session is received. A mean trajectory with error bounds is fitted to the test data. A state of the subject is determined by comparing the fitted mean trajectory with error bounds to a stored model of normality. The stored model of normality comprises a library of latent mean trajectories with error bounds. Each latent mean trajectory with error bounds is derived by fitting a hierarchical probabilistic model to a respective one of a plurality of sets of historical data. Each set of historical data comprises a plurality of session data units. Each session data unit representing a time-series of physiological measurements obtained during a different measurement session. The latent mean trajectory with error bounds for the set describes an underlying function governing each of the time-series of the session data units of the set.

The invention relates to monitoring a subject, particularly to detect a high risk of an adverse medical development in the subject, such as a high risk of intra-dialytic hypotension (IDH).

Each year, millions of people worldwide suffer from chronic long-term diseases such as diabetes, heart disease, and kidney malfunction, with limited access to appropriate treatment.

As a particular example, in patients with end-stage renal failure, 50% of patients require renal replacement therapy haemodialysis (HD). HD is a complex procedure that removes excess fluid from the blood stream, restores the balance of electrolytes, and removes waste products (such as urine) from the blood. HD is extremely stressful on the physiology of a patient, with high associated mortality rates. Patients are at risk of intra-dialytic hypotension (IDH; i.e., a sudden drop of blood pressure), which can lead to chronic heart disease and death. Blood pressure is recorded irregularly during a 4-hour session. Additionally, there are no standard definitions of IDH or other abnormal events in the HD setting, and care is often subjective and variable in efficacy.

Although there are approaches in the literature to classify the health of a patient according to their vital signs, most of them are either heuristic [1] or assumed the data are time invariant and independent [2]. For modelling time-series and the state of a patient, vital sign measurements commonly contain different numbers of observations, and at irregularly sampled times. To address these problems, Gaussian processes have been used for modelling physiological time series data [3], but further improvements are desirable.

It is an object of the invention to provide improved methods and apparatus for monitoring patients.

According to an aspect of the invention, there is provided a computer-implemented method of monitoring a human or animal subject, comprising: receiving test data representing a time-series of physiological measurements performed on a subject in a measurement session; fitting a mean trajectory with error bounds to the test data; and determining a state of the subject by comparing the fitted mean trajectory with error bounds to a stored model of normality, wherein: the stored model of normality comprises a library of latent mean trajectories with error bounds, each latent mean trajectory with error bounds being derived by fitting a hierarchical probabilistic model to a respective one of a plurality of sets of historical data; and each set of historical data comprises a plurality of session data units, each session data unit representing a time-series of physiological measurements obtained during a different measurement session, the latent mean trajectory with error bounds for the set describing an underlying function governing each of the time-series of the session data units of the set.

The method allows time-series measurement data to be classified efficiently and flexibly based on historical data. The method makes it possible to use information derived from large quantities of historical data (e.g. time-series data from many different measurement sessions) without requiring excessive computing resource and without sacrificing sensitivity, specificity or reliability. In particular, instead of trying to compare test data trajectories individually against corresponding time-series data obtained from every available similar time-series obtained previously, methods according to the present disclosure organise session data units into sets and use those sets to learn a latent mean trajectory with error bounds associated with the set. By comparing a fitted mean trajectory with error bounds representing the test data to a library of the latent mean trajectories with error bounds, a more computationally efficient comparison between the test data and the historical data is made possible.

The method thus makes it possible to identify patients at risk of an adverse medical development efficiently in a wider range of situations (e.g. without requiring massive computational resources to be accessible). Patient outcomes and quality-of-life between measurement sessions can be improved.

In an embodiment, the fitting of the mean trajectory with error bounds to the test data comprises fitting a Gaussian Process to the test data. Fitting of Gaussian Processes to time-series data is well-known and can be implemented efficiently.

In an embodiment, the fitting of the hierarchical probabilistic model to each set of historical data comprises fitting a Hierarchical Gaussian Process to each set of historical data, each latent mean trajectory with error bounds comprising a latent Gaussian Process. Fitting of Hierarchical Gaussian Processes to sets of time-series data can be implemented efficiently.

In an embodiment, each set of historical data comprises session data units obtained from a single subject, the subject being different for at least a subset of the sets of historical data. This approach provides a natural grouping of session data units that will be similar to each other when each subject is in the normal state. Grouping by subject also means that, where a subject has sufficient historical data that a reliable latent mean trajectory with error bounds for that subject is present in the library, that latent mean trajectory with error bounds may be used as an efficient point of comparison for determining whether test data from that subject indicates that the subject is in a normal state or an abnormal state.

In an embodiment, each set of historical data comprises session data units obtained exclusively from a plurality of subjects having a phenotype of interest in common, the phenotype of interest being different for at least a subset of the sets of historical data. This approach allows the method to potentially identify which phenotype cluster a subject belongs to based on test data from the subject, thereby obtaining additional information about the subject (i.e. the subject's phenotype), optionally in addition to determining whether the subject is in a normal state or an abnormal state.

According to an alternative aspect, there is provided an apparatus for monitoring a human or animal subject, comprising: a data receiving unit configured to receive test data representing a time-series of physiological measurements performed on a subject in a measurement session; a data processing unit configured to: fit a mean trajectory with error bounds to the test data; and determine a state of the subject by comparing the fitted mean trajectory with error bounds to a stored model of normality, wherein: the stored model of normality comprises a library of latent mean trajectories with error bounds, each latent mean trajectory with error bounds being derived by fitting a hierarchical probabilistic model to a respective one of a plurality of sets of historical data; and each set of historical data comprises a plurality of session data units, each session data unit representing a time-series of physiological measurements obtained during a different measurement session, the latent mean trajectory with error bounds for the set describing an underlying function governing each of the time-series of the session data units of the set.

Embodiments of the invention will now be described, by way of example only, with reference to the accompanying drawings in which corresponding reference symbols indicate corresponding parts, and in which:

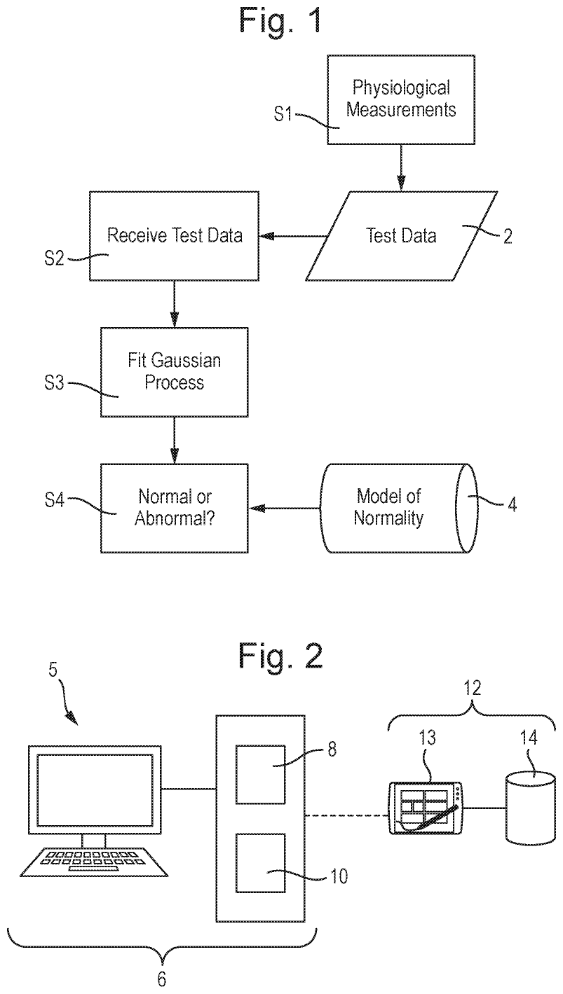

FIG. 1 schematically depicts a method of monitoring a subject according to an embodiment;

FIG. 2 schematically depicts an apparatus for monitoring a subject;

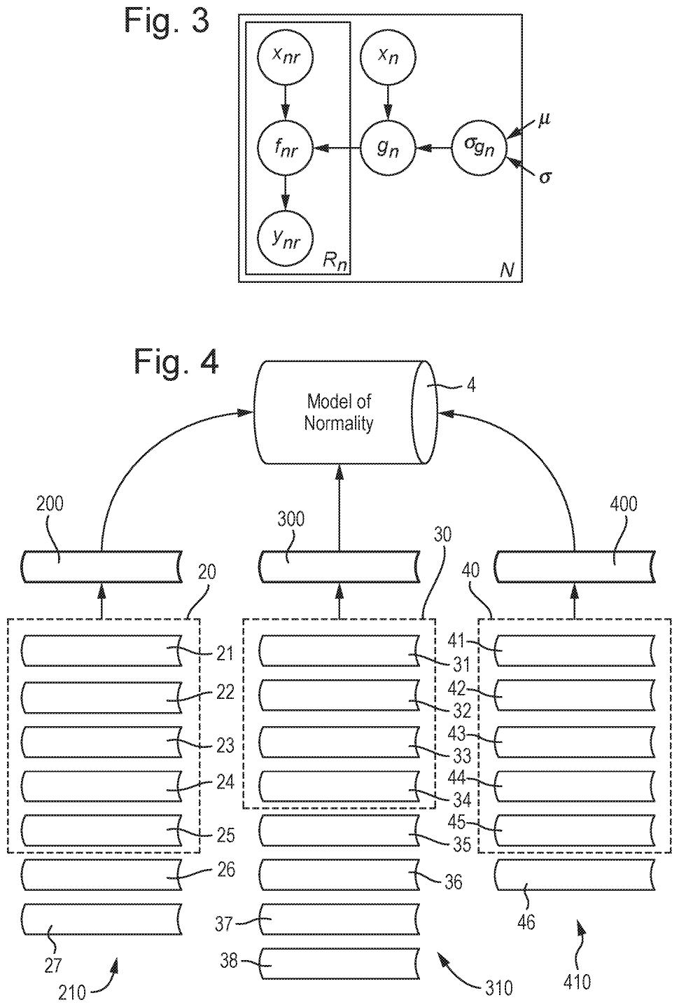

FIG. 3 is a graphical representation of a Bayesian Hierarchical Gaussian Process;

FIG. 4 schematically depicts training of a model of normality;

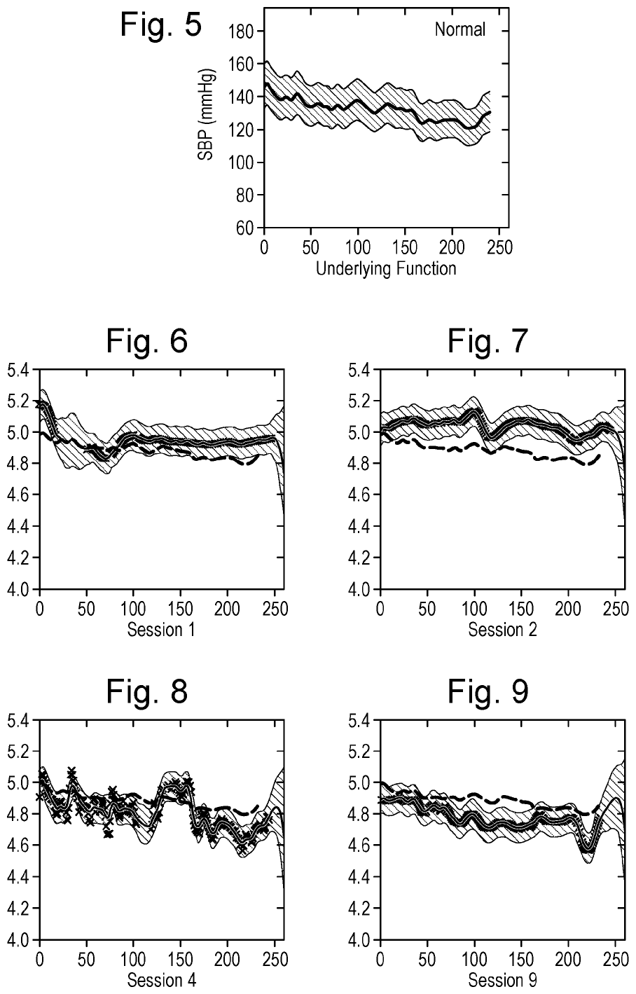

FIG. 5 depicts an inferred latent Gaussian Process for a subject derived from session data units from an SBP dataset (Example 1) corresponding to measurement sessions in which the patient was in a normal state;

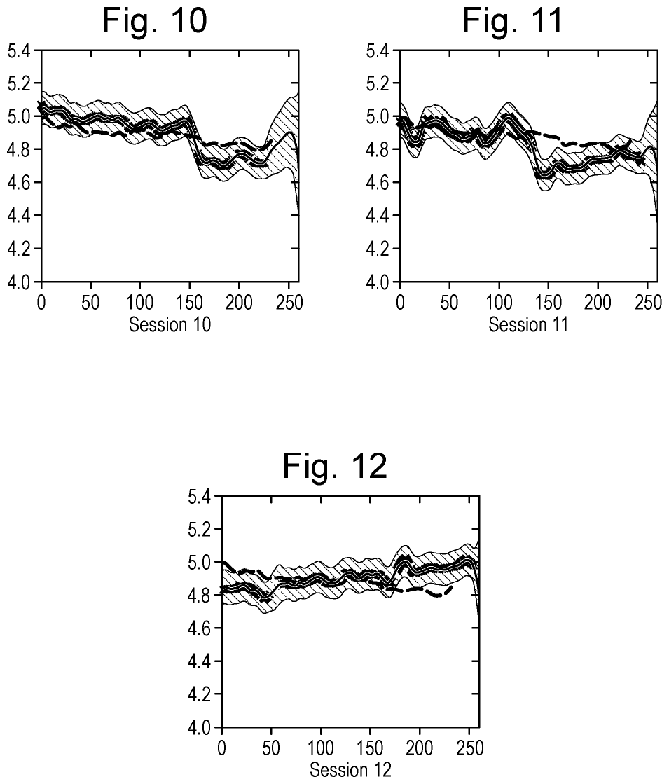

FIGS. 6-12 depict session data units used to derive the latent Gaussian Process of FIG. 5;

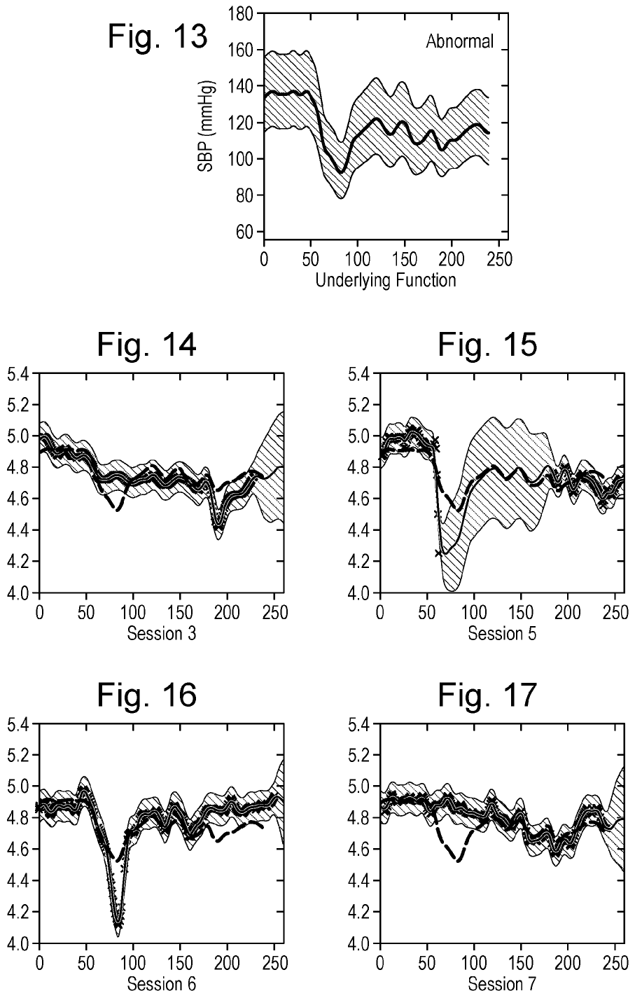

FIG. 13 depicts an inferred latent Gaussian Process for a subject derived from session data units from the SBP dataset (Example 1) corresponding to measurement sessions in which the patient was in an abnormal state;

FIGS. 14-17 depict session data units used to derive the latent Gaussian Process of FIG. 13;

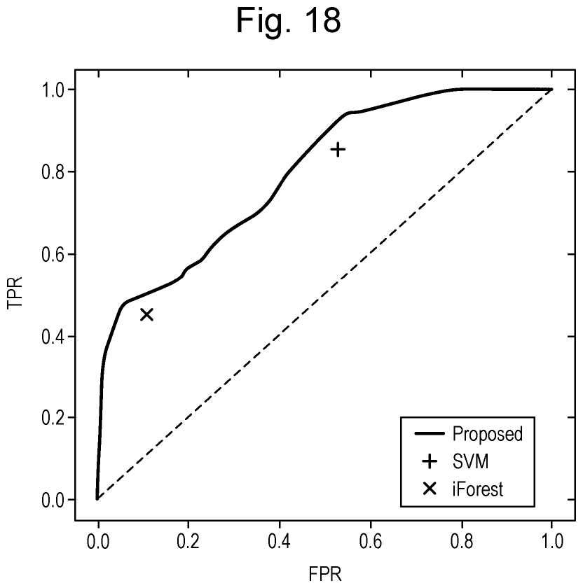

FIG. 18 depicts a Receiver-Operating-Characteristic (ROC) curve for multiple different values of D_{KL}_{ₘ} thresholds of normality for the SBP dataset (Example 1);

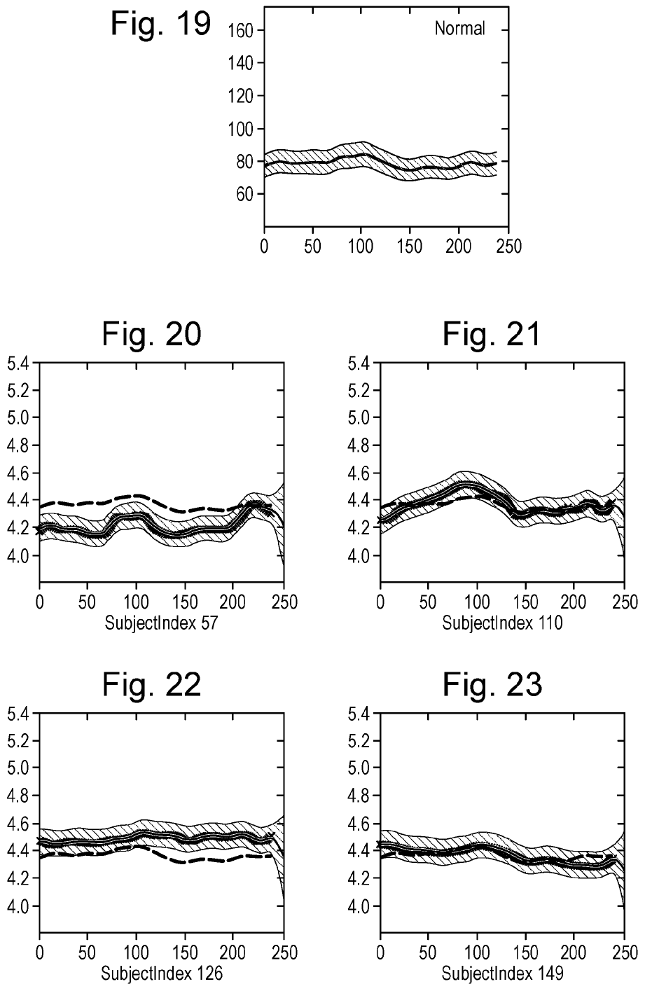

FIG. 19 depicts an inferred latent Gaussian Process for a cluster of subjects in an HR dataset (Example 2) in the normal state;

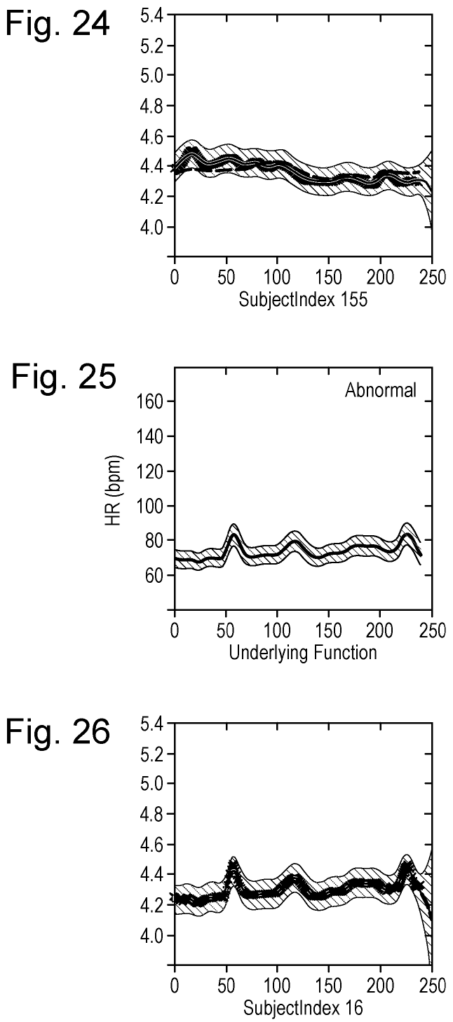

FIGS. 20-24 depict session data units for different subjects of the cluster corresponding to the inferred latent Gaussian Process of FIG. 19;

FIG. 25 depicts an inferred latent Gaussian Process for an abnormal subject in the HR dataset (Example 2);

FIG. 26 depicts a session data unit for the subject corresponding to the inferred latent Gaussian Process of FIG. 25; and

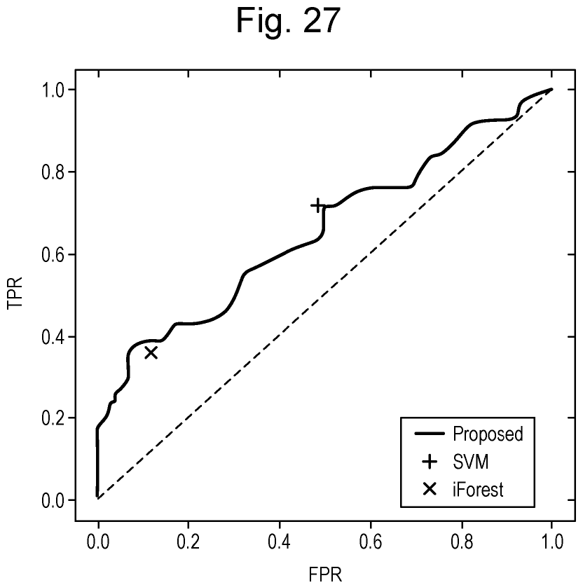

FIG. 27 depicts a Receiver-Operating-Characteristic (ROC) curve for multiple different values of D_{KL}_{ₘ} thresholds of normality for the HR dataset (Example 2).

Methods of the present disclosure are computer-implemented. Each step of the disclosed methods may therefore be performed by a computer. The computer may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, smart device (e.g. smart TV), etc. Alternatively, the computer may consist of a distributed computing system having plural different computers connected to each other via a network such as the internet or an intranet.

FIG. 1 schematically depicts a framework for methods of monitoring a human or animal subject according to embodiments of the disclosure. The methods may be performed by an apparatus 5 for monitoring a human or animal subject as depicted in FIG. 2. The terms “human or animal subject” or “subject” may be used interchangeably with the term “patient” in the following description.

The method comprises a step S1 of performing physiological measurements on a subject in a measurement session. The physiological measurements may be performed used a sensor system 12 as depicted schematically in FIG. 2. The sensor system 12 may comprise a local electronic unit 13 (e.g. a tablet computer, smart phone, smart watch, etc.) and a sensor unit 14 (e.g. a blood pressure monitor, heart rate monitor, etc.). The physiological measurements may comprise one or more vital signs measurements, including one or more of the following: blood pressure measurements, heart rate measurements, breathing rate measurements, temperature measurements, oxygen saturation measurements. In a first detailed embodiment discussed below, the physiological measurements comprise blood pressure measurements (systolic blood pressure, SBP). In a second detailed embodiment discussed below, the physiological measurements comprise heart rate (HR) measurements.

In step S2, test data 2 obtained by performing the physiological measurements in step S1 is received by a data receiving unit 8. The data receiving unit 8 may form part of a computing system 6 (e.g. laptop computer, desktop computer, etc.). The computing system 6 may further comprise a data processing unit 10 configured to carry out steps of the method.

In step S3, a mean trajectory with error bounds is fitted to the test data 2. The fitted mean trajectory with error bounds may comprise (or consist of) a fitted Gaussian Process.

In step S4, a state of the subject is determined. The determination of the state of the subject may comprise determining whether the subject is in a normal state or in an abnormal state. In some embodiments, the state of the subject is determined by comparing the fitted mean trajectory with error bounds (e.g. Gaussian Process) to a stored model of normality 4. The test data 2 can thereby be used to detect whether the subject is in an abnormal state, such that medical attention may be needed.

In some embodiments, the model of normality comprises a library of latent mean trajectories with error bounds (e.g. a library of latent Gaussian Processes). Each latent mean trajectory with error bounds is derived by fitting a hierarchical probabilistic model to a respective one of a plurality of sets of historical data. In some embodiments, the hierarchical probabilistic model comprises a Bayesian Hierarchical Gaussian Process (Bayesian HGP). Each set of historical data comprises a plurality of session data units. Each session data unit represents a time-series of physiological measurements obtained during a different measurement session. The latent mean trajectory with error bounds (e.g. latent Gaussian Process) for the set describes an underlying function governing all of the time-series of session data units of the set. This is described in further detail below in the case where the latest mean trajectory with error bounds is a latent Gaussian Process obtained by fitting a Bayesian HGP to a set of session data units.

In some embodiments, the determination of the state of the subject from the model of normality uses calculations of a metric of similarity, which may be performed by calculating a Kullbeck-Leibler divergence. Example implementations of Bayesian HGPs and calculations of Kullbeck-Leibler divergence are now described in detail.

### Bayesian Hierarchical Gaussian Processes

We assume that there are Rₙ time-series (such as physiological measurements over time), each corresponding to one of the session data units described above, for the nth subject, and they are similar to each other. The observed data for the Rₙ time-series can be defined

Yₙ={yₙᵣ}ᵣ₌₁^{R}^{ₙ}

taken at times

Tₙ={xₙᵣ}ᵣ₌₁^{R}^{ₙ}.

We further assume that there is a latent Gaussian Process (GP) function which governs all of the mean time-series for the nth subject, denoted as gₙ(x). Given a draw from gₙ, each r=1, . . . , Rₙ time-series of data for this nth subject is then drawn from a GP as:

fₙᵣ(x)˜GP(gₙ(x),k_{f}(x,x′)). (1)

The structure of Hierarchical Gaussian Processes (HGPs) for the nth subject is defined as [4]:

gₙ(x)˜GP(0,k_{g}(x,x′)), [parent structure]

fₙᵣ(x)˜GP(gₙ(x),k_{f}(x,x′)). [child structure] (2)

Within each subject's r time-series, the deviation of vital measurement from gₙ (x) is modelled by fₙᵣ, which is only affected by the parent structure via the subject's latent mean. Two data-points within time-series fₙᵣ(x) are jointly Gaussian distributed with zero mean and covariance k_{g}(x,x′)+k_{f}(x,x′). Two data-points in different time-series are, however, jointly distributed with covariance k_{g}(x,x′).

A graphical representation of Bayesian HGPs is shown in FIG. 3. yₙᵣ corresponds to the rth time-series for the nth subject and is modelled by the hidden node fₙᵣ given the observed timestamps xₙᵣ. The model further assumes there is a latent hidden GP, gₙ, which governs the relationship among {fₙᵣ}ᵣ₌₁^{R} over timestamps xₙ. A log-normal prior was placed over the variance hyperparameter (i.e., σ_{gn}) of gₙ.

The likelihood of our model can be described as:

$$p ( {Y}_{n} \vert {T}_{n} , θ ) = N ( {Y}_{n} \vert 0 , {\sum }_{n} ) , {\sum }_{n} [ r , {r}^{′} ] = \{ \begin{matrix}{K}_{g} ( {x}_{nr} , {x}_{{nr}^{′}} + & \\ {K}_{f} ( {x}_{nr} , {x}_{{nr}^{′}} ) + {σ}_{y}^{2} 𝕀 & if r = {r}^{′} \\ {K}_{g} ( {x}_{nr} , {x}_{{nr}^{′}} ) & otherwise\end{matrix} , \qquad ( 3 )$$

where [symbol] is the Kronecker delta function and σ_{y}² is the noise variance; K_{g}(xₙᵣ,x_{nr′}) denotes the covariance matrix for the latent “parent” structure, and its (i,j)^{th} element is estimated from the covariance function k_{g} (xₙᵣ, x_{nr′}) from the rth time-series for the nth subject. In a similar manner, K_{g}(xₙᵣ,xₙᵣ′) K_{f}(xₙᵣ,x_{nr′}) denotes the covariance matrix for the structure of separated GPs. The following covariance functions can be used:

$${k}_{g} ( {x}_{nr} , {x}_{{nr}^{′}} ) = \underbrace{{σ}_{1}^{2} ( 1 + \frac{\sqrt{5} r}{{λ}_{1}} + \frac{5 {r}^{2}}{3 {λ}_{1}^{2}} ) exp ( - \frac{\sqrt{5} r}{{λ}_{1}} )}_{{Matern }_{\frac{5}{2}} ( {σ}_{1} , {λ}_{1} )} \\ {k}_{f} ( {x}_{nr} , {x}_{{nr}^{′}} ) = \underbrace{{σ}_{2}^{2} ( 1 + \frac{\sqrt{3} r}{{λ}_{2}} ) exp ( - \frac{\sqrt{3} r}{{λ}_{2}} )}_{{Matern }_{\frac{3}{2}} ( {σ}_{2} , {λ}_{2} )} + \underbrace{{σ}_{3}^{2} exp ( - \frac{{r}^{2}}{2 {λ}_{3}^{2}} )}_{RBF ( {σ}_{3} , {λ}_{3} )} + \underbrace{{σ}_{f}^{2} 𝕀}_{noise} . \qquad ( 4 )$$

k_{g} has a Matern kernel for capturing short-term variability in a time-series trajectory. k_{f} is a noisy version of k_{g} containing RBF and Matern kernels for describing both long- and short-term variability. Hyper-parameters of the covariance functions can be estimated using type-II maximum-a-posteriori. Concretely, the probability model of the rth time-series of subject n is:

p(fₙᵣ*|xₙᵣ*,xₙᵣ,yₙᵣ)=N(μₙᵣ*,Σₙᵣ*),

μₙᵣ*=K_{gf}^{T}(xₙᵣ,xₙᵣ*)[K_{gf}(xₙᵣ,xₙᵣ)+σ_{y}² [symbol] ]⁻¹yₙᵣ

Σₙᵣ*=K_{gf}(xₙᵣ*,xₙᵣ*)

−K_{gf}^{T}(xₙᵣ,xₙᵣ*)[K_{gf}(xₙᵣ,xₙᵣ)+σ_{y}² [symbol] ]⁻¹K_{gf}(xₙᵣ,xₙᵣ*) (5)

where K_{gf}=K_{g}+K_{f}.

### A Metric for Similarity

The Kullback-Leibler (KL) divergence is commonly used as a measure of the non-symmetric difference between two continuous probability distributions P and Q as

$${D}_{KL} ( P \vert \vert Q ) = {\int }_{- ∞}^{∞} p ( x ) log \frac{p ( x )}{q ( x )} dx ,$$

where p and q denote the densities of P and Q. In the case where both P and Q are two multivariate normals (MVN) with dimension d, their KL divergence can be computed as:

$${D}_{KL} ( P \vert \vert Q ) = \frac{1}{2} [ ln \frac{\vert {\sum }_{Q} \vert}{\vert {\sum }_{P} \vert} - d + Tr ( {\sum }_{Q}^{- 1} {\sum }_{P} ) ] + \frac{1}{2} [ {( {μ}_{Q} - {μ}_{P} )}^{T} {\sum }_{Q}^{- 1} ( {μ}_{Q} - {μ}_{P} ) ] , \qquad ( 6 )$$

where μ_{Q} and Σ_{Q}, μ_{P} and Σ_{P} are the mean and covariance of P and Q respectively. Furthermore, the symmetric KL divergence as a metric for the similarity of two MVN distributions can be defined as D_{KL}=D_{KL}(P∥Q)+D_{KL}(Q∥P). The symmetric KL divergence is an example of a metric of similarity that can be used as a metric for classifying abnormal test data (e.g. SBP time-series or HR time series, as described below) from a model of normality.

### Constructing a Model of Normality

FIG. 4 schematically depicts a process for constructing a model of normality 4 usable in methods (e.g. in step S4 of FIG. 1) according to embodiments of the disclosure.

In an embodiment, a Bayesian HGP is fitted to each of a plurality of sets 20,30,40 (three in the example of FIG. 4) of historical data.

In some embodiments, each set of historical data comprises session data units obtained from a single subject. The subject corresponding to each set is different for at least a subset of the sets of historical data. Thus, in such an embodiment, the structure of FIG. 4 could be such that set 20 contains session data units 21-25 obtained exclusively from a first subject, set 30 contains session data units 31-34 obtained exclusively from a second subject, and set 40 contains session data units 41-45 obtained exclusively from a third subject.

In some embodiments, the plurality of sets 20,30,40 of historical data comprise a plurality of normal sets of historical data exclusively comprising session data units corresponding to measurement sessions in which the patient was in a normal state for the whole measurement session. Thus, in the example of FIG. 4, session data units 21-27 may have been obtained for the first subject, but the session data units 26 and 27 are excluded from the set 20 that is to be used to form the model of normality 4 because session data units 26 and 27 corresponded to measurement sessions in which an abnormal episode occurred (e.g. an intra-dialytic event occurred). Session data units 35-38 were excluded from set 30, and session data unit 46 was excluded from set 40, for corresponding reasons (because abnormal episodes were recorded in each corresponding measurement session). In some embodiments, the model of normality 4 is constructed exclusively using the plurality of normal sets of historical data. An alternative approach is to construct a model exclusively using abnormal data sets, but the greater variability in the underlying biology contributing to measurement sessions containing abnormal episodes (in comparison with normal behaviour) reduces accuracy.

In some embodiments, each of at least a subset of the sets of historical data comprises session data units obtained exclusively from plural subjects that are in the same one of a plurality of phenotype clusters. Each phenotype cluster contains subjects having a physiological characteristic in common corresponding to the phenotype. For example, in the case where the physiological measurements comprise measurements of blood pressure, the subjects may be clustered or phenotyped according to a propensity to have high blood pressure (a first phenotype), medium blood pressure (a second phenotype), low blood pressure (a third phenotype), etc., respectively. Applying this scenario to the structure of FIG. 4, instead of the session data units 21-27 all coming from the same subject, they could come from plural different subjects belonging to a first phenotype cluster, the session data units 31-38 could come from plural different subjects belonging to a second phenotype cluster, and the session data units 41-46 could come from plural different subjects belonging to a third phenotype cluster.

Whether or not the sets comprise session data units from single subjects or multiple subjects, the fitting of a Bayesian HGP to each set 20,30,40 allows a latent Gaussian Process 200,300,400 to be derived (e.g. as described above in “Bayesian Hierarchical Gaussian Processes”) for each set 20,30,40. The latent Gaussian Process 200,300,400 governs all of the time-series represented by the session data units of the set to which it corresponds. Each latent Gaussian Process may be thought of as an underlying function representing the true (hidden) characteristics of the subject or cluster of subjects to which the latent Gaussian Process corresponds. All session data units that are obtained when measuring that subject or cluster of subjects in the state corresponding to the set (e.g. in the normal state or the abnormal state) are thus related to the underlying function and generated thereby. By determining how similar a fitted Gaussian Process (fitted to test data) is to a latent Gaussian Process, it is possible to detect either or both of which set the test data belongs to (e.g. which cluster of subjects the subject being monitored is most similar to, which may be relevant for subsequent medical decisions or life-style choices) and whether the subject being monitored is in an abnormal state (and therefore possibly in need of urgent clinical attention).

In some embodiments, the comparison of the fitted Gaussian Process to the stored model of normality 4 comprises comparing the fitted Gaussian Process to each of one or more of the latent Gaussian Processes in the model of normality 4. In an embodiment, this comparison comprises calculating a metric of similarity between the fitted Gaussian Process and each of one or more of the latent Gaussian Processes in the model of normality 4.

In an embodiment, the metric of similarity is calculated for a latent Gaussian Process derived from a set of historical data obtained from the same subject as the test data. In this case, it is expected that the latent Gaussian Process should be a particularly good model of the subject because it is derived from historical measurements from the same subject (assuming that a sufficient amount of data is available).

In an embodiment, the metric of similarity is calculated for a latent Gaussian Process derived from a set of historical data corresponding to a cluster of subjects to which the subject providing the test data belongs. For example, in the case where the subject is known to be prone to high blood pressure, the metric of similarity may be calculated for the latent Gaussian Process corresponding to a set of historical data for a cluster of subjects prone to high blood pressure.

In an embodiment, the metric of similarity is calculated for each of a plurality of different latent Gaussian Processes. In such an embodiment, the plural resulting metrics of similarity may be used to indicate which one of a plurality of clusters of subjects the subject providing the test data belongs to. For example, if the metric of similarity is highest for a cluster corresponding to subjects prone to high blood pressure, the subject may be assigned to that cluster. The determination of the state of the subject may in this case comprise assigning the subject to one of a plurality of clusters. In the present example the determined state of the subject may thus be that the subject is prone to high blood pressure. The method may, however, go on to determine whether or not the subject is in a normal state or an abnormal state for that cluster (e.g. to determine that the subject is both prone to high blood pressure generally and currently in an abnormal state for the cluster of subjects that are prone to high blood pressure).

In some embodiments, the calculation of the metric of similarity comprises calculating a Kullback-Leibler divergence between the fitted Gaussian Process and the latent Gaussian Process, as described above.

In some embodiments, the determination of the state of the subject comprises comparing the calculated metric of similarity with a threshold. In an embodiment, the threshold is predetermined. In an embodiment, the threshold is obtained by calculating metrics of similarity for plural pairs of latent Gaussian Processes in the stored model of normality 4. This may for example provide a measure of expected variability between different subjects in the normal state and/or different clusters of subjects in the normal state. A detailed example is described below in the section “One-Class Classification”, in which a distribution of maximum normal KL values is used to derive appropriate thresholds for the comparison.

### DETAILED EXAMPLES/VALIDATION

The following describes further aspects of methods according to the disclosure in the context of two detailed examples: 1) using blood pressure monitoring data (SBP) from 35 subjects undergoing HD with measurement sessions taking place on different days; and 2) using heart rate (HR) vital-sign measurements from 336 subjects in a step-down unit.

### Data Description—Example 1 (SBP)

A total of 35 subjects (26 males and 9 females) were considered, with a median age of 60 (interquartile range of 51-76.5) years old. Detailed demographics of the subjects can be found in [5]. Continuous blood pressure monitoring (an example a physiological measurement) was performed using a Finometer device with each subject during routine HD treatment. Each HD visit lasted up to four hours, and there was a maximum of 3 sessions per week, resulting in a total of 3 to 27 HD sessions per individual depending on number of weeks that they were monitored. The definition of an abnormal session is as follows: one or more IDH events where the mean arterial blood pressure (MAP) was less than 60 mmHg as an indication of cerebral ischemia [5] and SBP less than 80% of the baseline SBP. Alternatively, MAP less than 60 mmHg was used to identify IDH events when there is no baseline SBP (i.e., BP prior to treatment). The beat-to-beat blood pressure data were processed by removal of extreme outliers (i.e., 3 or more standard deviations from the mean), and were low-pass filtered (cut-off frequency at f=0.083 Hz) to attenuate high frequency noise. An in-house signal quality metric based on waveform quality [5] was used to remove noisy segments. The measurements were then down-sampled minutely. Furthermore, we note that not all subjects have both normal (i.e., no IDH) and abnormal sessions (i.e., with IDH) throughout the study. A total of 156 normal sessions and 190 abnormal sessions were considered (noting that some subjects contributed both to the total of normal and to the total of abnormal sessions).

### Pre-Processing—Example 1 (SBP)

The minutely-sampled SBP data for each session of an individual subject was initially fitted with a univariate GP for the process of artefact removal. To accommodate both long-term and transient changes, the following covariance function was used:

$$k ( x , {x}^{′} ) + {σ}_{y}^{2} δ ( x , {x}^{′} ) = {Matern}_{\frac{5}{2}} + {RBF}_{s} + {RBF}_{l} + noise$$

where RBFₛ and RBFₗ refer to short and long length-scale variability in the RBF covariance function, respectively, and their length-scales were fixed to be λ=5 and λ=200 respectively. The length-scale of the

$${Matern}_{\frac{5}{2}}$$

was fixed to be λ=15. Furthermore, a log-uniform prior was used for each variance hyperparameter in the covariance functions. We note that the length scales are fixed to define the general trend of a session time-series, but its variance as a scaling factor is allowed to change as it determines variation of function values from their mean. Each session data was log-transformed and the mean was subtracted prior to fitting a GP. A moving window centred at each data point (±5 minutes) was used to compute the normalised mean LML for that data point with respect to the GP. The normalisation of the mean was performed using the standardised zero mean and unit variance approach. A threshold τ on LML was used to determine outliers, and any data points with LML below τ were deemed to be artefacts and were removed. The process was repeated in an iterative manner until 95% percent of data remained. We allowed the threshold values to vary in the range of τ=[−2.5, 1] for all subjects. After artefact removal, we fitted a univariate GP for each session using log-uniform priors over all hyperparameters.

### Data Description—Example 2 (HR)

The heart rate (HR) vital-sign measurements were extracted from 336 patients in a step-down unit at the University of Pittsburgh Medical Centre (UPMC) [9]. 112 clinical emergency events were identified by clinicians for 59 patients. These emergency events were defined to be any single period, at least several minutes in length, in which measurements from any vital-sign channel (HR, RR, SaO₂, BP) were “abnormally” high or low (using clinical definitions of abnormality [9]). For the purpose of this study, only the first emergency event for each of the 59 patients was considered, to avoid the confounding effect that may arise due to clinical intervention subsequent to an emergency event. We note that it could be the case that some of these events arose due to abnormality in a vital sign other than HR. However, we hypothesise that some of these non-HR events contain abnormal HR dynamics and thus our (HR-based) modelling will consider all 59 “first events” independent of which vital sign was the primary cause of the event.

We wish to compare the trajectories of data from these abnormal patient physiology to the trajectories of data from normal patients. Ostensibly, patient vital-sign dynamics are at their most “normal” in the period immediately before discharge. Data from normal patients were used if they were at least four hours of data before discharge; data from abnormal patients were used if there were at least four hours of data, centred around the annotated clinical event. These requirements were met by 170 normal patients and 47 abnormal patients.

### Pre-Processing—Example 2 (HR)

Each HR time-series was cleaned of transient artefacts using an artefact-detection algorithm that has been previously validated on the UPMC data set [10]. Unlike the GPR-based artefact detection method, described earlier, the artefact detection algorithm in [10] assumes data were independent-and-identically-distributed (iid) with respect to a short time window, τ=5 minutes, hence a unique 5-minute window was placed at each HR data point. A Gamma distribution was fitted to all HR measurements within the window, and the log-likelihood of each HR measurements was evaluated with respect to the fitted Gamma distribution. A measurement's artefact score was calculated as the average log-likelihood, across each 5-minute window containing that measurement. Artefacts with extremely low values are considered to be far away from other measurements close in time. These measurements were removed as they were likely to be artefacts.

### Clustering Sub-Populations—Example 2 (HR)

In order to test the use of the HGPs framework for inferring latent structure from a population of subjects, we identified clusters of sub-populations (corresponding to the phenotype clusters discussed above) within the set of normal patients. As the number of clusters is unknown, we applied the mixture-of-Gaussians framework proposed by Hensman et al. [11] to estimate the number of clusters using the truncated Dirichlet process prior. This procedure offers the advantage of finding the likely number of clusters in the data via the posterior Dirichlet distribution over the number of clusters. A total of 37 clusters were created from 170 normal subjects, and we then further removed clusters that combined a single HR time-series; this resulted in 32 groups from 165 subjects with between two and 14 subjects in each cluster. The resulting clusters are then used for subsequent analysis as described below.

### One-Class Classification

The normal data were considered to form a representative latent trajectory using Bayesian HGPs. For Example 1 (SBP), the process was performed for each subject, resulting in a total of 26 normal latent subject trajectories (e.g. latent Gaussian Processes), comprising 156 sessions. For Example 2 (HR), 32 clusters were created among normal subjects using the Mixture of Gaussians with variational inference [11] and HGPs was used to derive the latent trajectory to represent each cluster. Our model of normality 4 can then be used to classify abnormal sessions for the SBP data set and abnormal subjects for the HR data set, respectively.

1) Cross-Validation and Partitioning of Training and Test Sets: A leave-one-out (LOO) method was considered to train our model of normality 4 using N latent trajectories: At each fold, pairwise KL values (e.g. D_{KL} values as described above) were obtained (i.e., the MVN latent trajectory of a subject or a cluster was compared to others), and resulted in an N-by-N matrix of KL values. To estimate the threshold of the KL values to compare for classifying a trajectory as abnormal (corresponding to the threshold against which the calculated metrics of similarity referred to above are compared), we obtained the maximum of the normal KL values. We approached this by calculating the maximum normal KL value from each subject in the training set (denoted as D_{KL}_{ₘ} hereafter), indicating the most dissimilar behaviour within the normal population. From these D_{KL}_{ₘ} values, their mean and standard deviation were derived to define the distribution of D_{KL}_{ₘ}. Various KL thresholds of “normality” can then be defined (such as a range of ±3 standard deviation from the mean). Regarding the test set for the SBP dataset, we considered the normal sessions from the left-out subject, and all abnormal sessions. In order to create a balanced test set for both normal and abnormal data, we drew the same number of sessions from the abnormal population (i.e., 190 sessions) as those from the left-out subject. To make sure that we sampled from an adequate number of abnormal sessions, we repeated our draw randomly 100 times using the sampling-without replacement. For the HR data set, we followed the same methodology but using the normal subjects from the left-out cluster instead, and selected the same number of abnormal subjects from a total of 33 for testing.

2) Statistical measures: The predictive performance of each fold was assessed by sensitivity, specificity, and accuracy. Sensitivity was defined to be the proportion of abnormal trajectories (i.e. test data that resulted in the method indicating that the subject was in an abnormal state) that had been correctly identified as such. Specificity was used to measure the proportion of normal trajectories (i.e. test data that resulted in the method indicating that the subject was in a normal state) that had been correctly identified, and accuracy corresponded to the proportion of trajectories that had been correctly classified. The median sensitivity of the 100 draws was calculated for each fold, and the mean value across N folds was estimated for sensitivity, specificity, and accuracy. We further computed the Receiver-Operating-Characteristic (ROC) curve (shown in FIG. 18 for Example 1 and in FIG. 27 for Example 2) using the mean true positive rate (denoted as TPR as equivalent to sensitivity), and mean false positive rate (denoted as FPR as 1—specificity) for different D_{KL}_{ₘ} thresholds of normality.

### Method of Comparison

We compared our model with different clustering approaches, aiming to identify the structural difference between normal and abnormal sessions. The following benchmarking methods were considered: (1) k-means clustering (KMean), (2) Spectral Clustering (SClust) [6], (3) Balanced Iterative Reducing and Clustering using Hierarchies (BIRCH) [7], (4) Hierarchical clustering with Euclidean distance (HClust), (5) Mixture of Gaussians (MOG) with a Dirichlet process prior [4]. To further compare the robustness of our proposed one-class classification algorithm, we had compared its performance with the following novelty-detection methods for high-dimensional dataset: (1) One-class SVM with RBF kernel [12]; (2) Isolation Forest (iForest) [13]. Both methods were trained using five-fold cross validation by permuting the normal sessions. We assigned 20% of data for each fold of the cross validation as the hold-out test set. To obtain a balanced test set for both normal and abnormal data, we drew the same number of sessions from the abnormal population as those from the hold-out set, following the same method as described previously in our proposed algorithm.

### Results and Discussion

### SBP Dataset (Example 1)

### Inferring Latent Trajectories Using Bayesian HGPs

A total of 52 subject-specific latent trajectories were inferred using the HGPs model, of which 26 were normal and abnormal respectively. Each latent trajectory was fitted using the hierarchical structure described in Equations (2) and (4), and a log-normal prior (i.e., LN(μ=1, σ=0.25)) was used to regularise the variance (i.e., σ_{g}_{ₙ}) in the covariance function of the parent structure. Furthermore, the noise variance in a log-scale for the child structure (i.e., ln(σ_{f}²)) as well as that of the Bayesian HGPs model (i.e., ln(σ_{y}²)) were constrained with bound of [le-3,1] to prevent over-fitting. The parameters were estimated using the Python GPy package [8]. Ten random restarts were considered and the parameter values were estimated via maximum LML. Noting that the Bayesian HGPs model was fitted to each subject individually, and the latent trajectory was learnt from normal and abnormal sessions separately.

FIGS. 5-17 show the results of the latent (i.e., parent) trajectories of a subject inferred using Bayesian HGPs. The normal and abnormal latent underlying functions (latent Gaussian Processes) are demonstrated respectively in the two sets of figures: 1) FIGS. 5-12 and 2) FIGS. 13-17. The inferred latent structure of the SBP time-series gₙ(x) (i.e., underlying function or latent Gaussian Process) for the nth subject is plotted in FIG. 5 for normal behaviour and in FIG. 13 for abnormal behaviour, with 95% confidence interval. Each subsequent pane in each set of figures (i.e. FIGS. 6-12 and 13-17 respectively) is the rth session of the log-scaled SBP time-series fₙᵣ(x) with 95% confidence interval (i.e. each is a time-series corresponding to one of the session data units of the set of historical data corresponding to the latent Gaussian Process shown in FIG. 5 or 13). The session numbers are indicated in the bottom of each plot. The solid lines in each plot show the mean of the predicted function, and the shaded area represents the 95% confidence interval. The broken line in FIGS. 5-12 and 14-17 represents the latent structure in log-scale. Times on the horizontal axes are measured in minutes as the amount of time elapsed since the start of the HD session (an example of a measurement session).

Although the measurement sessions contain different numbers of measurements and have different times, we can leverage the advantage of the GP over other regression methods, and provide prediction that is robust to missing values. The confidence interval of a GP also provides a probabilistic interpretation of the model accuracy. Furthermore, upon improving on a univariate GP model by assuming each session is independent, the Bayesian HGPs model not only can infer a latent structure from multiple correlated sessions, but also provide an adequate fitting to each individual session, as its covariance structure is composed of both parent and child structure (i.e., K_{g}+K_{f}). In the two latent trajectories, our model was more confident in the normal case as their sessions were more closely related to each other within similar magnitudes. While in the abnormal case, only two out of four sessions were similar at 50 to 100 minutes period (see sessions 5 and 6 in FIGS. 15 and 16 respectively), and the other two had a small decrease in the period of 150 to 200 minutes (see sessions 3 and 7 in FIGS. 14 and 17 respectively). These disagreements are expected as the human physiology is dynamic throughout the HD treatment, and we anticipate that a patient may deteriorate at different timestamps for different HD sessions. Nevertheless, the Bayesian HGPs model was able to describe a larger uncertainty as a result of the disagreement among sessions, and demonstrated with a larger confidence interval for indicating possible abnormality that occurred at different time-stamps. As there are large variants in the abnormal sessions, we utilised only the normal latent trajectories to create a model of normality, and then classify if a test session is abnormal (as a form of “outliers”) using the symmetric KL divergence.

### Clustering HD Sessions

A fundamental problem in clustering is the handling of missing values, as imputation of missing values can be time-consuming and erroneous. However, GP regression handles missing values problems easily. For a fair comparison using the same dataset, we considered the mean function outputs from Bayesian HGPs as the data for each session, and we combined all normal and abnormal sessions together for performing unsupervised clustering (with number of clusters fixed to be two). The results of the bench-marking methods are reported in Table I below. It was interesting to observe that all methods had approximately 50% accuracy, demonstrating the difficulty for separating normal from abnormal sessions. These methods also assumed the data-points in each timeseries were independent, which limit their use for time-series clustering. In addition, these unsupervised methods only considered the mean function outputs of the Bayesian HGPs time-series for each session, and ignored the covariance matrix (i.e., the function that describes the confidence interval of each time-series), further reducing their abilities to separate normal from abnormal population. Therefore, we proceed with learning the latent trajectories of normal subjects and created a model of normality, with the goal to improve on our classification results of identifying abnormal sessions.

TABLE I

| | Statistical measures | | |
|---|---|---|---|
| Method | Sensitivity | Specificity | Accuracy |
| KMean | 0.70 | 0.31 | 0.52 |
| SClust | 0.30 | 0.72 | 0.49 |
| BIRCH | 0.66 | 0.40 | 0.54 |
| HClust | 0.63 | 0.39 | 0.52 |
| MOG | 0.58 | 0.37 | 0.48 |

### One-Class Classification

Out of the 26-fold LOO cross-validation, the mean and standard derivation of the log(D_{KL}_{ₘ}) were obtained as 8.30±0.11 and 1.01±0.03. They have demonstrated that the KL values were consistent among the latent trajectories with little variation. We then performed the statistical measures by varying the threshold within the range of [5.2, 11.4] (i.e., ±3 standard deviation from the mean in the log(D_{KL}_{ₘ}) distribution), as shown in FIG. 18. Furthermore, the Area-Under-Curve of our proposed algorithm is 0.80, with an accuracy of 0.71±0.07 at threshold value of log(D_{KL}_{ₘ})=9.22, which is higher than the bench-marking algorithms. This is higher than the bench-marking algorithms, where the accuracy of one-class SVM and iForest is 0.66±0.06 and 0.67±0.01, respectively. To further improve performance, our algorithm allows the threshold of log(D_{KL}_{ₘ}) to vary, and provides the flexibility of choosing a user-defined sensitivity and specificity.

### HR Dataset (Example 2)

### Inferring Latent Trajectories Using Bayesian HGPs

A total of 32 latent HR trajectories were inferred using the HGPs model, from 32 clusters of normal subjects. Each latent trajectory was fitted using the HGPs structure as described as before.

FIGS. 19-24 demonstrate the normal latent trajectory of a cluster inferred using HGPs. The inferred latent structure of the HR time-series gₙ(x) (i.e., underlying function) for the nth cluster is plotted in FIG. 19 with 95% confidence interval, and each subsequent panel (FIGS. 20-24) is the rth subject of the log-scaled HR time-series f_{iw}(x) with 95% confidence interval. FIGS. 25 and 26 show an example of the latent structure of an abnormal subject learnt using HGPs. The subject numbers are indicated in the bottom of each plot (in FIGS. 19-26). The normal and abnormal behaviours are learnt separately. The solid lines in each plot show the mean of the predicted function, and the shaded area represents the 95% confidence interval. The dotted line represents the latent structure in log-scale. The times on the horizontal axes are measured in minutes as the last four hours of discharge.

### Clustering HR Subjects

We had considered the mean function outputs from HGPs as the data for each subject, and stacked all normal and abnormal subjects together for performing unsupervised clustering (with number of clusters fixed to be two). The results of the performance for each method are reported in Table II. It was interesting to observe that non-linear methods had approximately 50% accuracy, demonstrating the difficulty for separating normal from abnormal subjects. As discussed before, these methods assumed the data-points in each time-series were independent, which limit their use for time-series clustering.

TABLE II

| | Statistical measures | | |
|---|---|---|---|
| Method | Sensitivity | Specificity | Accuracy |
| KMean | 0.49 | 0.43 | 0.44 |
| SClust | 0.49 | 0.62 | 0.59 |
| BIRCH | 0.54 | 0.47 | 0.52 |
| HClust | 0.54 | 0.47 | 0.52 |
| MOG | 0.72 | 0.19 | 0.31 |

### One-Class Classification

From the 32-fold LOO cross-validation, the mean and standard derivation of the log(D_{KL}_{ₘ}) were estimated to be 5.25±0.03 and 0.47±0.01. These KL values show that the latent trajectories were very similar with little variation. We then performed the statistical measures by varying the threshold within the range of [0,15] as shown in FIG. 27. Furthermore, we compared our results again with the bench marking algorithms, one-class SVM and iForest. It was shown that our method outperformed the iForest but was sub-optimal when compared with the one-class SVM. The Area-Under-Curve of our proposed algorithm is 0.65, with an accuracy of 0.65±0.06 at threshold value of log(D_{KL}_{ₘ})=9.36. This is similar to the bench-marking algorithms, where the accuracy of one-class SVM and iForest is 0.62±0.07 and 0.62±0.02, respectively. The results were expected as normal and abnormal subjects exhibited similar HR trajectories in both magnitude and trend, making it very challenging to distinguish the difference.

### REFERENCES

[1] H. Gao, A. McDonnell, D. A. Harrison, T. Moore, S. Adam, K. Daly, L. Esmonde, D. R. Goldhill, G. J. Parry, A. Rashidian et al., “Systematic review and evaluation of physiological track and trigger warning systems for identifying at-risk patients on the ward,” Intensive care medicine, vol. 33, no. 4, pp. 667-679, 2007.
[2] L. Tarassenko, A. Hann, and D. Young, “Integrated monitoring and analysis for early warning of patient deterioration,” British Journal of Anaesthesia, vol. 97, no. 1, pp. 64-68, 2006.
[3] L. Clifton, D. A. Clifton, M. A. Pimentel, P. J. Watkinson, and L. Tarassenko, “Gaussian processes for personalized e-health monitoring with wearable sensors,” IEEE Transactions on Biomedical Engineering, vol. 60, no. 1, pp. 193-197, 2013.
[4] J. Hensman, N. D. Lawrence, and M. Rattray, “Hierarchical Bayesian modelling of gene expression time series across irregularly sampled replicates and clusters,” BMC bioinformatics, vol. 14, no. 1, pp. 1 12, 2013.
[5] C. MacEwen, “Can data fusion techniques predict adverse physiological events during haemodialysis?” Ph.D. dissertation, University of Oxford, 2016.
[6] J. Shi and J. Malik, “Normalized Cuts and Image Segmentation,” IEEE Transactions on Pattern Analysis and Machine Intelligence, vol. 22, no. 8, pp. 888-905, Aug. 2000.
[7] T. Zhang, R. Ramakrishnan, and M. Livny, “BIRCH: an efficient data clustering method for very large databases,” in ACM Sigmod Record, vol. 25, no. 2. ACM, 1996, pp. 103-114. [8] GPy, “GPy: A Gaussian process framework in Python,” http://github.com/SheffieldML/GPy, since 2012.
[9] A. Hann, “Multi-parameter monitoring for early warning of patient deterioration,” Ph.D. dissertation, University of Oxford, 2008.
[10] G. Colopy et al., “Likelihood-based artefact detection in continuously acquired patient vital signs,” 39th Intl. Conf of the IEEE EMB Society (EMBC), Jeju, South Korea, 2017.
[11] J. Hensman, N. D. Lawrence, and M. Rattray, “Hierarchical Bayesian modelling of gene expression time series across irregularly sampled replicates and clusters,” BMC bioinformatics, vol. 14, no. 1, pp. 1 12, 2013.
[12] C. C. Chang and C. J. Lin, “LIBSVM: A Library for Support Vector Machines,” ACM Transactions on Intelligent Systems and Technology, vol. 2, no. 3, pp. 27:1-27:27, May 2011.
[13] F. T. Liu, K. M. Ting, and Z.-H. Zhou, “Isolation-Based Anomaly Detection,” ACM Transactions on Knowledge Discovery from Data, vol. 6, no. 1, pp. 3:1-3:39, Mar. 2012.

1. A computer-implemented method of monitoring a human or animal subject, comprising:
receiving test data representing a time-series of physiological measurements performed on a subject in a measurement session;
fitting a mean trajectory with error bounds to the test data; and
determining a state of the subject by comparing the fitted mean trajectory with error bounds to a stored model of normality, wherein:
the stored model of normality comprises a library of latent mean trajectories with error bounds, each latent mean trajectory with error bounds being derived by fitting a hierarchical probabilistic model to a respective one of a plurality of sets of historical data; and
each set of historical data comprises a plurality of session data units, each session data unit representing a time-series of physiological measurements obtained during a different measurement session, the latent mean trajectory with error bounds for the set describing an underlying function governing each of the time-series of the session data units of the set.

2. The method of claim 1, wherein the fitting of the mean trajectory with error bounds to the test data comprises fitting a Gaussian Process to the test data.

3. The method of claim 2, wherein the fitting of the hierarchical probabilistic model to each set of historical data comprises fitting a Hierarchical Gaussian Process to each set of historical data, each latent mean trajectory with error bounds comprising a latent Gaussian Process.

4. The method of claim 1, wherein the comparison of the fitted mean trajectory with error bounds to the stored model of normality comprises comparing the fitted mean trajectory with error bounds to each of one or more of the latent mean trajectories with error bounds in the library.

5. The method of claim 1, wherein each set of historical data comprises session data units obtained from a single subject, the subject being different for at least a subset of the sets of historical data.

6. The method of claim 1, wherein each set of historical data comprises session data units obtained exclusively from a plurality of subjects having a phenotype of interest in common, the phenotype of interest being different for at least a subset of the sets of historical data.

7. The method of claim 1, wherein the plurality of sets of historical data comprise:
a plurality of normal sets of historical data exclusively comprising session data units corresponding to measurement sessions in which the subject was in a normal state for the whole measurement session; and
the model of normality is constructed exclusively using the plurality of normal sets of historical data.

8. The method of claim 1, wherein the comparison of the fitted mean trajectory with error bounds to the stored model of normality comprises calculating a metric of similarity between the fitted mean trajectory with error bounds and each of one or more of the latent mean trajectories with error bounds in the library.

9. The method of claim 8, wherein the metric of similarity is calculated for a latent mean trajectory with error bounds derived from a set of historical data obtained from the same subject as the test data.

10. The method of claim 8, wherein the calculation of the metric of similarity comprises calculating a Kullback-Leibler divergence between the fitted mean trajectory with error bounds and the latent mean trajectory with error bounds.

11. The method of claim 8, wherein the determination of the state of the subject comprises comparing the calculated metric of similarity with a threshold.

12. The method of claim 11, wherein the threshold is obtained based on a distribution of calculated metrics of similarity for plural pairs of latent mean trajectories with error bounds in the library.

13. The method of claim 1, wherein the physiological measurements comprise one or more of the following: blood pressure measurements, heart rate measurements, breathing rate measurements, temperature measurements, oxygen saturation measurements.

14. The method of claim 1, wherein at least a subset of the measurement sessions used to train the stored model, for each of one or more of the subjects, are performed on different days.

15. The method of claim 1, wherein the measurement sessions comprise haemodialysis sessions.

16. The method of claim 15, wherein each session data unit was determined to correspond to a measurement session in which the patient was in an abnormal state during at least part of the measurement session if one or more of the following is observed:
one or more intra-dialytic events was observed during the measurement session, optionally defined as where one or both of: the mean arterial blood pressure is less than 60 mmHg as an indication of cerebral ischemia and systolic blood pressure is less than 80% of a baseline systolic blood pressure.

17. A computer program comprising computer-readable instructions that cause a computer to perform the method of claim 1.

18. A computer program product storing the computer program of claim 17.

19. An apparatus for monitoring a human or animal subject, comprising:
a data receiving unit configured to receive test data representing a time-series of physiological measurements performed on a subject in a measurement session;
a data processing unit configured to:
fit a mean trajectory with error bounds to the test data; and
determine a state of the subject by comparing the fitted mean trajectory with error bounds to a stored model of normality,
wherein:
the stored model of normality comprises a library of latent mean trajectories with error bounds, each latent mean trajectory with error bounds being derived by fitting a hierarchical probabilistic model to a respective one of a plurality of sets of historical data; and
each set of historical data comprises a plurality of session data units, each session data unit representing a time-series of physiological measurements obtained during a different measurement session, the latent mean trajectory with error bounds for the set describing an underlying function governing each of the time-series of the session data units of the set.

20. The device of claim 19, further comprising a sensor system configured to perform the physiological measurements on the patient to provide the test data.
